(19) United States
(12) Patent Application Publication
(10) Pub. No.: US 2007/0037893 A1
(43) Pub. Date: Feb. 15, 2007

(54) Process to transport a methanol or hydrocarbon product

(76) Inventor: Stuart Ritchie Bradford, London (GB)

Correspondence Address: SHELL OIL COMPANY, P O BOX 2463, HOUSTON, TX, 772522463, US

(21) Appl. No.: 10/577,568

(22) PCT Filed: Oct. 27, 2004
(86) PCT No.: PCT/EP04/52679

§ 371 Date: Apr. 27, 2006

(30) Foreign Application Priority Data
Oct. 29, 2003 (EP) 03256832.1

(51) Int. Cl. C07C 27/26

(52) U.S. Cl. 518/726

(57) ABSTRACT

A process to transport a hydrocarbon product from one location to another location by means of a ship wherein the hydrocarbon product is obtained by: (a) separating air into oxygen and nitrogen; (b) using the oxygen to prepare a mixture of carbon monoxide and hydrogen from a carbonaceous source; (c) using the mixture of carbon monoxide and hydrogen to prepare a liquid or solid hydrocarbon product; wherein the process comprises loading the liquid or solid hydrocarbon product in a ship together with the nitrogen as obtained in step (a).

Many publications are known describing processes for the conversion of gaseous hydrocarbonaceous feed stocks, as methane, natural gas and/or associated gas, into liquid products, especially methanol and liquid or solid hydrocarbons, particularly paraffinic hydrocarbons. In this respect often reference is made to remote locations (e.g. in the dessert, tropical rain-forest) and/or offshore locations, where no direct use of the gas is possible, usually due to the absence of large populations and/or the absence of any industry. Transportation of the gas, e.g. through a pipeline or in the form of liquefied natural gas, requires extremely high capital expenditure or is simply not practical. This holds even more in the case of relatively small gas production rates and/or fields. Reinjection of gas will add to the costs of oil production, and may, in the case of associated gas, result in undesired effects on the crude oil production. Burning of associated gas has become an undesired option in view of depletion of hydrocarbon sources and air pollution. The present invention aims at providing a practical process of transportation of methanol or hydrocarbon products made from the gas at a remote location to a location to close to the (end) users of said methanol or hydrocarbon products.

The present invention relates to a process to transport a methanol or hydrocarbon product from one location to another location by means of a ship wherein the methanol or hydrocarbon product is obtained by,

(a) separating air into oxygen and nitrogen,

(b) use of said oxygen to prepare a mixture of carbon monoxide and hydrogen from a carbonaceous source,

(c) use of said mixture of carbon monoxide and hydrogen to prepare methanol or a liquid or solid hydrocarbon product, and wherein the ship is obtained by,

(d) loading said methanol or liquid or solid hydrocarbon product in the ship together with the nitrogen as obtained in step (a).

Step (a) is preferably performed by means of cooling air and isolating the liquid air components oxygen and nitrogen and optionally other components. The oxygen/nitrogen mixture used in step (a) is preferably air. Suitably, the stream enriched in oxygen contains at least 50 mol %, more suitably 85 mol % oxygen, based on the total stream, preferably 95 mol %, more preferably 98 mol %. Suitably the oxygen depleted stream contains at least 95 mol % nitrogen based on the total stream, preferably 98 mol %, more preferably 99 mol %. The oxygen depleted stream contains at most 2 mol % oxygen based on the total stream, preferably at most 1 mol %, more preferably at most 0.2 mol %. If desired, all traces of oxygen may be removed.

Cryogenic concepts have been developed over the years to liquefy and separate air into its main constituents nitrogen, oxygen and rare gases. Refrigeration for cryogenic applications is produced by absorbing or extracting heat at low temperature and rejecting it to the atmosphere at higher temperatures. Three general methods for producing cryogenic refrigeration in large-scale commercial application are the liquid vaporisation cycle, the Joule-Thomson expansion cycle and the engine expansion cycle. The first two are similar in that they both utilise irreversible isenthalpic expansion of a fluid, usually through a valve. Expansion in an engine approaches reversible isenthalpic expansion with the performance of work. For more detailed discussion reference is made to Perry's Chemical Engineers Handbook, Sixth Edition, 12-49 ff. (McGraw-Hill, New York, 1984), Kirk-Othmer, Encyclopedia of Chemical Technology, Fourth Edition, Volume 7, p. 662 ff. (John Wiley and Sons, New York, 1993) and Ullmann's Encyclopedia of Industrial Chemistry, Fifth Edition, Volume A 18, p. 332 ff. (VCH, Weinheim, 1991).

Most commercial air separation plants are based on Linde's double distillation column process. This process is clearly described in the above references. In a typical example, feed air is filtered and compressed to a pressure usually between 5 and 10 bara. The compressed air is cooled and any condensed water is removed in a separator. To avoid freezing of water and carbon dioxide in the cryogenic part of the plant, the feed air is further passed through an adsorbent bed, usually activated alumina and/or molecular sieves, to remove the last traces of water and carbon dioxide. The purified air is than cooled down further, and fed to a first cryogenic distillation unit, usually at an intermediate stage. Crude liquid material from the bottom section of the first distillation unit, usually comprising between 40 and 50 mol percent oxygen, is fed to the second distillation unit (which second unit is usually on the top of the first distillation unit, the condenser of the first column usually acting as the reboiler for the second unit), usually also at an intermediate stage. The second distillation unit is operated at relatively low pressure (usually 1 to 2 bara). At the top of the first distillation unit almost pure liquid nitrogen is obtained which is typically fed to the second column at the top. Pure liquid oxygen is obtained at the bottom of the second distillation unit, while pure gaseous nitrogen is obtained from the top of the second column.

Many variations on the above concept are known. These include separation of air into gaseous products, liquid products and all kind of combinations thereof. Also the production of partly enriched oxygen and/or nitrogen streams together with almost pure oxygen and/or nitrogen streams, either in liquid or gaseous phase is well known. In addition there may be additional distillation units to separate any of the rare gases present in the feed air. Further, the methods for creating the low temperatures may vary in many ways. In this respect reference is made to the above cited literature references, and further to EP-A-798524, JP-A-08094245, EP-A-593703, EP-A-562893, U.S. Pat. No. 5,237,822, JP-A-02052980, EP-A-211957, EP-A-102190, SU-A-947595 JP-A-71020126 and JP-A-71020125.

In step (b) the oxygen as obtained in step (a) is used for the production of a mixture of carbon monoxide and hydrogen, also referred to as synthesis gas. The carbonaceous feed to be used in the present process is suitably methane, natural gas, associated gas or a mixture of C₁₋₄ hydrocarbons, preferably associated gas, more preferably associated gas at a remote location. Other possible carbonaceous feedstocks are coal, brown coal, peat, heavy hydrocarbons, e.g. crude oil residues, e.g. pitch, and asphaltenes, and bio fuel, e.g. wood, organic waste products and vegetable oils.

Step (b) is preferably performed by means of a so-called partial oxidation. The partial oxidation may be carried out in an oxidation or gasification reactor. A well known process for the partial oxidation of a (hydro) carbonaceous feed is the Shell Gasification Process in which the (hydro)carbonaceous feed is partially combusted in a non-catalytic process at elevated temperature and pressure. In another embodiment the oxidation is carried out in the presence of a catalyst. Such catalysts are well known in the art and usually comprise one or more noble Group VIII metals. Steam and/or carbon dioxide may be added to the hydrocarbonaceous feed stream in order to adjust the H₂/CO ratio. The oxidation is suitably carried out at temperatures between 900 and 1500° C., preferably 1000 to 1350° C., and a pressure between 5 and 120 bar, especially between 25 and 70 bar. Typically the gaseous mixture has an H₂/CO ratio between 1:1 and 3:1, preferably about 2:1. Prior to contacting the gaseous mixture with a catalyst in step (c), it is preferred to remove compounds which could adversely effect the catalyst. In this respect reference is made to the removal of sulphur containing compounds and nitrogen containing compounds (e.g. NH₃ and HCN).

The purified gaseous mixture, comprising predominantly hydrogen and carbon monoxide, is used in step (c) to prepare the liquid or solid product or precursor to the product to be transported in the claimed process.

The product may suitably be methanol. Examples of processes to carry out step (c) to prepare methanol from carbon monoxide and hydrogen are well known and described in for example For example the ICI (Imperial Chemical Industries) process, the Lurgi process, and the Mitsubishi process may be used for step (c). In such processes the methanol synthesis gas is fed to a methanol synthesis reactor at the desired pressure of about 700 to 2000 psig, depending upon the process employed. The syngas then reacts with a copper based catalyst to form methanol. The reaction is exothermic. Therefore, heat removal is ordinarily required. The raw or impure methanol is then condensed and purified to remove impurities such as higher alcohols including ethanol, propanol, and the like. The uncondensed vapor phase comprising unreacted methanol syngas is recycled to the step (c).

In another embodiment according the invention step (c) is performed by contacting synthesis gas of step (b) with a catalyst, by which these compounds are converted into liquid or solid paraffins. The catalysts used for the catalytic conversion of the mixture comprising hydrogen and carbon monoxide into paraffinic hydrocarbons are known in the art and are usually referred to as Fischer-Tropsch catalysts. Catalysts for use in this process frequently comprise, as the catalytically active component, a metal from Group VIII of the Periodic Table of Elements. Particular catalytically active metals include ruthenium, iron, cobalt and nickel. Cobalt is a preferred catalytically active metal.

Examples of suitable Fischer-Tropsch synthesis processes for step (c) ate for example the so-called commercial Sasol process, the Shell Middle Distillate Process or by the non-commercial Exxon process. These and other processes are for example described in more detail in EP-A-776959, EP-A-668342, U.S. Pat. No. 4,943,672, U.S. Pat. No. 5,059,299, WO-A-9934917 and WO-A-9920720 and are incorporated by reference. The Fischer-Tropsch process may be carried out in a slurry reactor, a fixed bed reactor, especially a multitubular fixed bed reactor or in a three phase fluidised bed reactor.

The waxy product as prepared in the Fischer-Tropsch synthesis step may be transported as such according to the present process or transported as separate fractions. Suitably the Fischer-Tropsch synthesis product is subjected to a mild hydroisomerisation to reduce the congealing point of the product and increase its pumpability. The resulting synthetic crude may be shipped to a different location to be further worked up by traditional refining methods.

From the paraffin waxy product different grades of wax may be isolated at the remote location having congealing points between 25 and 120° C. Also lower boiling liquid fractions may be isolated from the waxy Fischer-Tropsch product boiling between 35 and 300° C. which may be shipped as hydrocarbon solvents, as steam cracker feedstock or as feedstock for the preparation of detergents.

Alternatively the waxy product is subjected to a hydrocracking/hydroisomerisation process wherein lower boiling fractions are obtained, such as for example paraffin products boiling in the naphtha, kerosene and gas oil boiling range. The partly isomerised liquid products so obtained may be shipped to end costumers for use as aviation fuel (blending components), diesel fuel (blending components), industrial gas oil (blending components), drilling fluids, steam cracker feedstock or solvents. The partly isomerised wax as obtained in such process steps may advantageously be further processed by means of dewaxing to obtain lubricating base oils or may be shipped as an intermediate product to base oil manufacturing locations more near to the end users. Examples of such processes are described in more detail in U.S. Pat. No. 6,309,432, U.S. Pat. No. 6,296,757, U.S. Pat. No. 5,689,031, EP-A-668342, EP-A-583836, U.S. Pat. No. 6,420,618, WO-A-02070631, WO-A-02070629, WO-A-02070627, WO-A-02064710 and WO-A-02070630, which references are incorporated by reference. The referred to hydrocracking/hydroisomerisation and optimal dewaxing steps are thus performed at the remote location and the resulting above described products are the hydrocarbon products to be shipped.

Step (d) is preferably performed by first purging the empty product containers in the ship with nitrogen as obtained in step (a) in order to lower the oxygen content. Purging is preferably performed for at least 5 minutes and more preferably for at least 10 minutes. Most preferably purging takes between 50 and 100 minutes. After purging the product containers are filled with the liquid or solid methanol or hydrocarbon product. Preferably nitrogen as obtained from step (a) is supplied to the loaded containers to achieve a nitrogen atmosphere in the gaseous space above the product in the product containers. More preferably nitrogen is supplied for at least 5 minutes and more preferably for at least 10 minutes. Typically nitrogen is supplied for not more than 20 minutes in order to minimise the loading operation. The pressure of the nitrogen used in step (d) is preferably above 2 bar, more preferably between 5 and 25 bar, and even more preferably between 15 and 20 bars.

The process according the present invention is especially suited for the specialities products and the solid products as obtained in step (b). Examples of such products are the detergent feedstock products, the base oil products, the partly isomerised wax products, the synthetic crude product and the wax products. Preferably the invention is applied to products which are transported as a liquid and/or to products which require liquefaction at loading and unloading. More preferably these products have a flash point of above 200° C.

It has been found that the advantages of using nitrogen in the present process are even more pronounced when the time in which the products are on board the ship is greater than 7 days and even more preferably on the ship for a period of greater than 30 days and up to 100 days.

Nitrogen, optionally stored as liquid nitrogen in the Fischer-Tropsch facility, may be used for many applications, such as for example as buffer gas for Compressor Dry Gas Seals, blanketing of drums during sampling of Fischer-Tropsch derived wax, inerting of equipment during unloading/loading of Fischer-Tropsch catalyst and hydroprocessing catalyst, cooling and inerting of various reactors, purging of idle wax lines, preservation of equipment or as means to maintain sufficient gas velocities during turndown operations of burner equipment of for example the burners of the partial oxidation equipment. It was however unexpected that this nitrogen could also be used so beneficial for the process according to the present invention.

1. A process to transport a methanol or hydrocarbon product from one location to another location by means of a ship wherein the methanol or hydrocarbon product is obtained by,
(a) separating air into oxygen and nitrogen;
(b) using said oxygen to prepare a mixture of carbon monoxide and hydrogen from a carbonaceous source;
(c) using said mixture of carbon monoxide and hydrogen to prepare methanol or a liquid or solid hydrocarbon product, wherein said process comprises loading said methanol or liquid or solid hydrocarbon product in a ship together with the nitrogen as obtained in step (a).

2. (canceled)

3. A process according to claim 1, in which a stream enriched in oxygen contains at least 85 mol % oxygen based on the total stream.

4. A process according to claim 1, in which an oxygen depleted stream contains at least 95 mol % nitrogen based on the total stream.

5. A process according to claim 1, wherein the product is methanol.

6. A process according to claim 1, wherein the hydrocarbon product is a paraffinic product as obtained in a Fischer-Tropsch process.

7. A process according to claim 1, wherein the loading is performed such that first nitrogen from step (a) is used to purge product containers on board the ship, secondly filling the product containers with the hydrocarbon product obtained in step (c) and subsequently adding an additional amount of nitrogen from step (a) to the product containers on board the ship.

8. A process according to claim 3, in which the stream enriched in oxygen contains at least 98 mol % oxygen.

9. A process according to claim 4, in which the oxygen depleted stream contains at least 99 mol % nitrogen.

10. A process to transport a methanol or hydrocarbon product from one location to another by means of a ship wherein the methanol or hydrocarbon product is obtained by.
(a) separating air into an oxygen enriched stream containing at least 85 mol % oxygen and an oxygen depleted stream containing at least 95 mol % nitrogen;
(b) using said oxygen enriched stream to prepare a mixture of carbon monoxide and hydrogen from a carbonaceous source;
(c) using said mixture of carbon monoxide and hydrogen to prepare a methanol or hydrocarbon product;
wherein said process comprises loading said methanol or hydrocarbon product onto a ship together with the nitrogen as obtained in step (a).

11. A process according to claim 10, in which the oxygen enriched stream contains at least 98 mol % oxygen.

12. A process according to claim 10, in which the oxygen depleted stream contains at least 99 mol % nitrogen.

13. A process according to claim 10, wherein the product is methanol.

14. A process according to claim 10, wherein the product is a liquid hydrocarbon.

15. A process according to claim 10, wherein the product is a solid hydrocarbon.

16. A process according to claim 10, wherein the hydrocarbon product is a paraffinic product obtained in a Fischer-Tropsch process.

17. A process according to claim 10, wherein said loading comprises:
purging a product container on the ship with nitrogen from step (a); and
filling the container with the methanol or hydrocarbon product.

18. A process according to claim 17, further comprising adding an additional amount of nitrogen from step (a) to the container after filling with methanol or hydrocarbon product.
